(19) United States
(12) Patent Application Publication
(10) Pub. No.: US 2008/0274046 A1
(43) Pub. Date: Nov. 6, 2008

(54) Radiolabelling

(76) Inventor: Christian Prenant, Manchester (GB)

Correspondence Address: NIXON & VANDERHYE, PC, 901 NORTH GLEBE ROAD, 11TH FLOOR, ARLINGTON, VA, 22203, US

(21) Appl. No.: 12/071,387

(22) Filed: Feb. 20, 2008

Related U.S. Application Data
(60) Provisional application No. 60/902,060, filed on Feb. 20, 2007.

(51) Int. Cl. A61K 51/10; C07C 47/24; A61K 51/08; A61K 101/02; A61K 51/04; C07C 45/00

(52) U.S. Cl. 424/1.49; 568/490; 568/495; 424/1.69; 424/1.73

(57) ABSTRACT

Compounds of the formula (I) are disclosed:

¹⁸F—(CHR)ₙ(CH₂)ₘCHO (I)

in which n and m are independently 0 and 1 with at least one of n and m being 1, and R (if present) is a hydrogen atom or a methyl group, subject to the proviso that if n is 1 and R is methyl then m is 0. Synthesis of the compounds is described together with their use in radiolabelling reactions, e.g. for the radiolabelling of peptides to facilitate detection by Positron Emission Tomography (PET) imaging.

The preferred compound is [¹⁸F]Fluoroacetaldehyde.

### FIELD OF INVENTION

The present invention relates to radiolabelling with ¹⁸F, i.e. a particular radioactive isotope of fluorine having a half-life about 110 min. The invention relates more specifically to compounds containing ¹⁸F, methods for the synthesis of such compounds, radiolabelled adducts prepared with such compounds and the use of such adducts in radio-imaging procedures.

The invention relates more particularly (but not necessarily exclusively) to ¹⁸F-containing compounds that may be used for the synthesis of radiolabelled probe molecules, in which case the ¹⁸F isotope provides for a ready means of detecting such probe molecules, particularly for the case where they are administered in vivo and require detection at a site targeted by the probe. Examples of such probes include radiolabelled peptides, proteins, antibodies, antibody fragments and oligonucleotides which may, for example, be used for Positron Emission Tomography (PET) imaging. A further possibility is the use of the compounds for radiolabelling pharmaceuticals. Further information on these subjects is provided by Journal Articles included in the “References” section of this specification.

### BACKGROUND

One particular example of ¹⁸F-radiolabelled probe molecule is a ¹⁸F-labelled RGD peptide which may be used for PET imaging, for example, tumour-induced, integrin associated angiogenesis.

A range of radiofluorination techniques have been applied to RDG peptides radiolabelling over the past few years. For example, direct electrophilic radiofluorination of a c[RGDf(NMe)V] with [¹⁸F]AcOF (Ogawa et al., 2003) yielding a fluorinated peptide with low specific radioactivity; [¹⁸F]fluoropropionylation of a glycosylated RGD-containing peptide c[RGDyK(SAA)] (Haubner et al., 2001 and 2004); oxime formation between an aminooxy-functionalized peptide and [¹⁸F]fluorobenzaldehyde under acidic condition (Poethko et al., 2004); [¹⁸F]fluorobenzoylation of a c(RGDyK) peptide via N-succinimidyl 4-[¹⁸F]fluorobenzoate (SFB method) (Chen et al., 2004) and a selective conjugation of a N-[2-(4-¹⁸F-Fluorobenzamido)Ethyl]Maleimide to the thiol group of a cystein derivatized c(RGDyK) peptide and its dimer E[c(RGDyK)]₂ (Cai et al., 2006). Although this latter technique has the advantage of the chemoselectivity for a thiol function it is like the SFB method not straightforward, time consuming and difficult to automate thus not easy to implement in routine production.

It is an object of the present invention to obviate or mitigate the above mentioned disadvantages

### SUMMARY OF INVENTION

The present invention provides compounds of the formula (I):

¹⁸F—(CHR)ₙ(CH₂)ₘCHO (I)

in which n and m are independently 0 and 1 with at least one of n and m being 1, and R (if present) is a hydrogen atom or a methyl group, subject to the proviso that if n is 1 and R is methyl then m is 0

Formula (I) embraces 2-[¹⁸F]fluoroacetaldehyde (n=0, m=1) and two [¹⁸F]-substituted propionaldehydes (n=m=1 and R═H; and m=0, n=1 and R=Me). Being aldehydes, the compounds of formula (I) react readily with compounds (eg macromolecules) having primary and secondary amino groups. The reaction may be effected under low temperature, mild conditions in organic or aqueous solvents (eg pH about 7) and the aldehydes may be used for the radiolabelling of a wide range of compounds containing such amino groups, particularly macromolecules such as peptides, proteins, antibodies, antibody fragments and oligonucleotides which should ideally not be subjected to more extreme conditions. It is particularly preferred that the reaction is effected under reductive amination so as to form a stable, derivative. Such reductive aminations may be effected in situ with reaction of the aldehyde and amino group under similarly mild conditions (in organic or aqueous solvents). This procedure provides a route to radiolabelled probe molecules for use in PET imaging.

Additionally the compounds of formula (I) can be produced quickly and easily by straightforward synthetic procedures which therefore makes them readily available for use in radiolabelling applications.

The preferred aldehyde of the invention is 2-[¹⁸F]fluoroacetaldehyde.

The aldehydes of the invention have a number of advantages for the purposes of readiolabelling. Specifically, aldehydes are more reactive than ketones in reductive alkylation reactions. Protein ethylation using acetaldehyde is almost as efficient as reaction with formaldehyde (the most reactive molecule used for reductive alkylation). Replacement of a hydrogen atom of acetaldehyde methyl group for a fluorine atom has no steric effect (Van der Waals radius of fluorine is similar to hydrogen) but a strong electronic effect as the fluorine atom has the highest electronegativity of all elements.

The small size of the fluoroethyl group (generated from the 2-[¹⁸F]fluoroacetaldehyde) has a minimal impact on peptide or protein integrity. Additionally the ability of the fluorine lone electron pair to participate readily in hydrogen bonding might mimic the ε-amino group of lysine which in proteins is usually involved in hydrogen bonding. As a result of its small Van der Waals radius (smallest after hydrogen) and its high electronegativity fluorine can substitute hydrogen atoms or hydroxyl groups of physiologically active compounds which can be dealt with similarly by biological systems (e.g. FDG where a hydroxyl is replaced by a fluorine). Furthermore due to the strong C—F bond energy, physiologically active fluorinated compounds can be resistant to metabolic degradation but similarly recognized.

Additionally, ¹⁸F has a physical half-life of 109.7 min and decay by 97% positron emission with low maximum energy (Emax β⁺=0.635 MeV). The shorter half-life and lower energetic positron emission compared with ¹²⁴I, contributes to a lower radiation burden to the patient.

The invention also provides a straight forward and simple to automate route to the compounds of formula (I) which are therefore made readily available for radiolabelling purposes. This method comprises the steps of:

(a) providing a compound of the formula (II).

¹⁸F—(CHR)ₙ(CH₂)ₘ-L (II)
in which m, n and R are as defined above and L represents a leaving group;
   (b) oxidising the compound of formula (II) to the aldehyde (I); and
   (c) recovering the aldehyde (I) from the reaction mixture.

### DESCRIPTION OF DRAWINGS

FIG. 1 is an HPC analysis trace (U.V. detection) for the Test Synthesis product mixture (Example 1) involving reaction of [¹⁸F]Fluoroacetaldehyde with benzylamine under reductive amination conditions.

FIG. 2 is a radioactivity trace of the same product mixture.

FIG. 3 is an HPLC analysis of the same product mixture but co-injected with N-(2-Fluoroethyl)-benzylamine as reference.

FIG. 4 is a reference trace for N-(2-Fluoroethyl)-benzyamine.

FIG. 5 is an HPLC analysis (radioactivity detection) of the product obtained by reaction of [¹⁸F]Fluoroacetaldehyde and cyclic RGDyK peptide.

FIG. 6 is an HPLC analysis (U.V. detection) of the reaction product of [¹⁸F]Fluoroacetaldehyde and cyclic RGDyK peptide.

FIGS. 7a and 7b show an HPLC analysis for the radiolabelling of an apoptosis biomarker with [¹⁸F]Fluoroacetaldehyde using radioactivity and U.V. detection respectively (Example 2).

FIGS. 8a and 8b are HPLC traces for the radiolabelling of an anti-VEGF antibody with [¹⁸F]Fluoroacetaldehyde using radioactivity and U.V. detection respectively (Example 3).

FIGS. 9a and 9b are HPLC traces for the radiolabelling of a 17 KDa peptide with [¹⁸F]Fluoroacetaldehyde using radioactivity and U.V. detection respectively (Example 5).

FIG. 10 shows autoradiographies of a binding assay using the radiolabelled 17 KDa peptide (Example 6).

### DESCRIPTION OF PREFERRED EMBODIMENTS

The invention provides new compounds of the formula (I) as defined above, the preferred compound being 2-[¹⁸F]fluoroacetaldehyde.

These aldehydes may be produced by a process which includes the steps of:

(a) preparing a compound of the formula (II).

¹⁸F—(CHR)ₙ(CH₂)ₘ-L (II)
in which m, n and R are as defined above and L represents a leaving group;
   (b) oxidising the compound of formula (II) to the aldehyde (I); and
   (c) recovering the aldehyde (I) from the reaction mixture.

Compound (II) may be prepared by means of a nucleophilic attack of the ¹⁸F— anion on an appropriate substrate for which examples are given below.

The ¹⁸F⁻ anion may be provided by an alkali metal salt of the formula ¹⁸F⁻ (M being an alkali metal). Such salts may be prepared from an aqueous [¹⁸F]fluoride solution which may itself be generated via the ¹⁸O(p,n)¹⁸F nuclear reaction by proton bombardment of an isotopically enriched [¹⁸O] water target, e.g. by use of an isochronous cyclotron such as a Scanditronix model MC60 PF. The alkali metal fluoride may be obtained by treatment of the aqueous [¹⁸F]fluoride solution with an alkali metal salt, e.g. the carbonate. For the purposes of the subsequent synthetic steps for obtaining the aldehydes (which will generally be carried out in an organic solvent), it is preferred that a chelating agent is incorporated in the solution containing the alkali metal fluoride. Subsequently water may be evaporated to provide the dry, complexed alkali metal fluoride for use in the subsequent steps of the procedure.

Examples of chelating agents include
a. Polyether rings with repeating (—CH₂CH₂O) units known as crown ethers such as: 1,4,7,10,13,16-Hexaoxacyclooctadecane (18-Crown-6) and substituted derivatives (more suitable for potassium cation) or 1,4,7,10,13-Pentaoxacyclopentadecane (15-Crown-5) and substituted derivatives (more suitable for sodium cation) or 1,4,7,10-Tetraoxacyclododecane (12-Crown-4) and substituted derivatives (specific for the lithium cation., 98%).
b. Bi or tricyclic cryptands, with stronger binding properties, containing nitrogen or sulphur together with oxygen atoms such as the aminopolyether 4,7,13,16,21,24-Hexaoxa-1,10-diazabicyclo[8.8.8]hexacosane (Kryptofix® 222, more suitable for potassium cation) or its alkyl and aryl derivatives.

The preferred alkali metal employed for the purposes of synthesis of the aldehydes is potassium although other alkali metals (e.g. sodium) may also be used.

In a first preferred synthetic method (Method I) in accordance with the invention for obtaining aldehydes (I), step (a) of the synthesis (ie preparation of compound of formula (II) —¹⁸F—(CHR)ₙ(CH₂)ₘ-L) is effected by reaction of the ¹⁸F— anion with a compound of the formula (III):

L-(CHR)ₙ(CH₂)ₘ-L (III)

in which m, n, L and R are as defined above.

The reaction may be a solution phase reaction between the compound of formula (III) with the alkali metal fluoride (prepared as described above), the reaction medium being a polar organic solvent. The solvent may for example be acetonitrile or DMSO. Mono-substitution of the compound of formula (III) may be achieved by appropriate stoichiometry of the reactants. Generally only nanomolar amounts of [¹⁸F] are produced by the cyclotron and may be reacted with a substantial molar excess of the compound of formula (III) so that double substitution is unlikely to occur. The reaction proceeds with mild conditions (eg a temperature of 80-90 C) and good yield. In subsequent step (b) of this preferred synthetic route (ie oxidation of compound (II)) to the desired aldehyde is affected by heating compound (II) in anhydrous DMSO which is a well established oxidation technique (Kornblum W, 1959). Oxidation may be achieved by heating the mixture of DMSO and compound (III) to a temperature of ca 140-160° C.

Conveniently recovery of the aldehyde is by distillation from the product mixture.

If DMSO was used as the solvent for the preparation of compound (II) then isolation of (II) is not required for the purposes of the oxidation reaction in DMSO. We have however found that use of acetonitrile as solvent for preparation of (II) (with subsequent recovery of (II), eg by evaporation of acetonitrile, prior to dissolution in DMSO) provides an improved overall yield.

In this preferred synthetic procedure (ie Method 1), the leaving group L is most preferably a tosylate group but there are other possibilities. These additional possibilities include halogen (preferably bromide or iodide), p-bromobenzenesulfonate or brosylate, p-nitrobenzenesulfonate or nosylate, methanesulfonate or mesylate, nonafluorobutane sulfonate or nonaflate, 2,2,2-trifluoroethanesulfonates or tresylate, trifluoromethane sulfonate or triflate, triflate and tresylate are least favoured as they contain stable fluorine atoms which could exchange with ¹⁸F.

The preferred overall synthetic route for Method 1 as applied to the production of [¹⁸F]fluoroacetaldehyde may therefore be depicted as

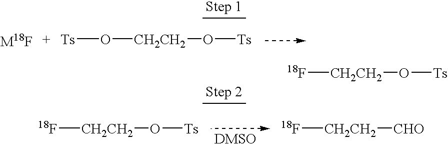

Step 1 is preferably effected in acetonitrile which is evaporated prior to dissolution of the product (of Step 1) in DMSO for the purposes of Step 2.

The mechanism of the DMSO oxidation is probably as follows:

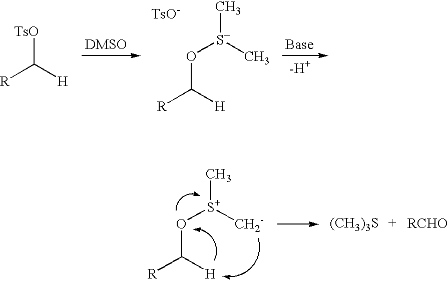

A base is effectively needed for the reaction as tosylate (TsO—) is not basic enough to trap the proton. We have however found that, if the M¹⁸F was prepared with potassium carbonate (see above) or other base then the residual carbonate is sufficient to act as base for the DMSO oxidation without the need for additional base. In an alternative synthetic procedure (Method 2) as applied to the synthesis of [¹⁸F]fluoroacetaldehyde, step (a) may be effected by the reaction of ethylene oxide or a propylene oxide (depending on the aldehyde to be produced) in solution with the alkali metal fluoride in the presence of a trapping agent to produce the compound (III). The trapping agent may, for example, be a carbodiimide such as N,N′-Dicyclohexylcarbodiimide (C₆H₁₁N═C═NC₆H₁₁). In the case of ethylene oxide as the starting material, the intermediate has the following structure:

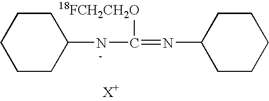

The counter ion X⁺ may for example be an alkali metal (e.g. complexed) or a tetraalkylammonium species. This compound is a urea derivative which is an activated alcohol similar to the tosylate derivative mentioned above. The urea derivative may then be oxidised in the same way as the tosylate derivative (e.g. oxidation in DMSO with or without addition of alkali metal bicarbonate) to produce the desired product which may then be distilled from the reaction mixture. It is however necessary to ensure that any excess alkylene oxide present in the reaction mixture is removed or destroyed since otherwise it may react with the amino groups of a compound to be labelled more efficiently than the aldehyde. The ethylene oxide and propylene oxide have low boiling points and may be removed by heating the reaction mixture to below 90° C., to avoid oxidation of the urea intermediate, with nitrogen bubbling.

The aldehydes of formula 1 may be used for radiolabelling a wide range of compounds, particularly those including a primary or secondary amino group. The aldehydes are particularly useful for the generation of radio labelled peptides, anti bodies, antibody fragments and oligonucleotides for PET imaging. The aldehydes react with the primary and secondary amino groups in both organic and aqueous solvents at low temperatures. These conditions are suitable for specifically lysine moieties of peptides and proteins.

The radiolabelling reaction is preferably conducted using reductive amination conditions which lead to the formation of a stable secondary amine. A suitable reagent for effecting the reductive amination is sodium cyanoborohydride.

The aldehydes (I) allow the reductive amination reactions to be effected under mild conditions, eg in aqueous media at about pH 7 using conventional buffers.

In a particular convenient way of effecting the radio labelling reaction, the aldehyde is distilled from its product mixture directly into a sample of the compound (e.g. in aqueous solution) to be radio labelled, the sample also including the necessary reagents to provide the reductive amination conditions. The aldehyde may for example be distilled (e.g. in a nitrogen stream) into a aqueous solution of the compound to be radiolabelled. However in an advantageous embodiment of the invention, the compound to be radiolablled is contained within a gel preferably an aqueous gel and/or most preferably a gel comprised of cross-linked polysaccharides. We have found this to be advantageous distilling the aldehyde into an aqueous solution of certain types of compounds (e.g. proteins) results in foaming which decreases the trapping efficiency for the aldehyde and also the overall yield of radiolabelled product.

Examples of gels that may be used include those based on dextran (e.g. G-25 and G-50) and Sephadex® (e.g. G-10, G-25, G-50, G-75 and G-100). Sephadex® G-75 is particularly preferred due to its porosity and swelling properties.

If the radiolabelling reaction is to be effected under reductive amination conditions then the reducing agent (e.g. sodium cyanoborohydride) may be incorporated in the gel.

The radiolabelled adduct may be prepared from an RDG peptide, eg E[c(RGDyK)]₂ or c(RGDyK). A further example for adduct formation is avastin.

Although the aldehydes of the invention are particularly useful for the production of radiolabelled probes they do have other applications. Thus, for example, [¹⁸F]Fluoroacetaldehyde can find application in Strecker amino acid synthesis (alanine analogue) and also in Mannich reactions.

The Mannich reaction consists of a amino alkylation of an acidic proton placed next to a carbonyl functional group with formaldehyde (or sometimes another aldehyde) and ammonia or any primary or secondary amine. The final product is a P-amino-carbonyl.

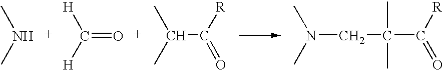

The invention is illustrated by the following non-limiting Examples.

### EXAMPLE 1

### General

Chemicals and solvents were purchased from Sigma-Aldrich Company Ltd. (Gillingham, UK) and were used without further purification. The aldehyde colorimetric test kit (formaldehyde-test) was purchased from Merck Chemicals Ltd. (Nottingham, UK). HPLC solvents and a ready-to-use commercial cyanoborohydride phosphate buffered saline solution (Cyanoborohydride Coupling Buffer) were obtained from Sigma. The coupling solution is made up of 0.02 M sodium phosphate, pH 7.5, containing 0.2 M sodium chloride and 3.0 g/L sodium cyanoborohydride. Wheaton borosilicate screw-top V-vials, capacity 3.0 mL and 1 mL, with open-top cap and PTFE faced silicon septum and heat transfer block from Aldrich were used for the radiosyntheses.

Thin layer chromatographies (TLC) were performed on Fluka silica gel plates (20×20 cm, 250 μm thickness). Radio-TLC plates were analysed with an instant imager (Packard).

Analytical high performance liquid chromatography (HPLC) was carried out on a Varian ProStar 210 solvent delivery system equipped with a semi-preparative C18 column (μBondapak™, 10 μm particle size; 300×7.8 mm i.d.; Waters Ltd., Elstree, UK). The eluate was monitored for absorbance (254 nm; model 325; Varian) and simultaneously for radioactivity with a Bioscan PM Flow-count detector. Collected radioactive peaks were measured on a Capintec CRC-15R.

¹H NMR spectra were recorded on a Bruker DPX 400 MHz spectrometer Tetramethylsilane was used as internal reference. ¹⁹F NMR spectra were recorded on a Bruker DPX200 MHz spectrometer.

Mass spectra (electrospray) were acquired on a Micromass Quattro II LC/MS.

### (i) Preparation of [¹⁸F]Fluoroacetaldehyde

[¹⁸F]fluoride was produced via the ¹⁸O(p,n)¹⁸F nuclear reaction by 17 MeV proton bombardment of an isotopically enriched [¹⁸O] water target (95-97% H₂¹⁸O water enrichment) using an isochronous cyclotron Scanditronix model MC60 PF.

[¹⁸F]potassium fluoride. The aqueous [¹⁸F]fluoride solution (0.3-0.5 ml, 100-150 MBq) was added to an open 3 mL conical vial containing potassium carbonate (1-1.7 mg, 7.2-12.3 μmol) and Kryptofix® 222 (10 mg, 26.6 μmol). After addition of 1 mL of acetonitrile the mixture was dried under argon at 100° C. This drying step was repeated twice with 1 mL of acetonitrile.

2-[¹⁸F]Fluoroethyltosylate. To the dried Kryptofix/[¹⁸F]fluoride complex was added a solution of ethylene di(p-toluenesulfonate), 5 mg (13.5 μmol) in 0.3-0.4 mL acetonitrile, the vial was sealed and the mixture was heated for 8 min at 90° C. 2-[¹⁸F]Fluoroethyltosylate was produced with 75 to 88% yield (radio-TLC: chloroform-methanol, 96:4, R_{f} 0.82).

[¹⁸F]Fluoroacetaldehyde. The evaporation of the acetonitrile at 50-70° C., under nitrogen, was followed by addition of 0.2-0.25 mL of anhydrous DMSO and [¹⁸F]Fluoroacetaldehyde was distilled, as the oxidation reaction proceeded at 150° C. for 4 min then 130° C. for another 4 min, and conveyed by a current of nitrogen (7-8 ml/min) into the second reaction vial.

A check by gas chromatography analysis using a flame ioniation detector (FID) demonstrated that no glyoxal was distilled with the [¹⁸F]fluoroacetaldehyde. (Since ethylene di-p-toluenesulfonate was used in excess for the production of 2-[¹⁸F]fluoroethyl p-toluenesulfonate (compound II, L=tosylate), glyoxal was a potential product of ethylene di-p-toluenesulfonate (compound III, L=tosylate) oxidation with DMSO. Glyoxal is a volatile liquid (boiling point=51° C.) which is readily soluble in water and which reacts with amines. Its production and distillation with [¹⁸F]fluoroacetaldehyde would compromise the use of the latter as a N—[¹⁸F]fluoroethylating agent).

### (ii) Characterisation of [¹⁸F]Fluoroacetaldehyde

This characterisation involves comparison of N-(2-fluoroethyl)-benzylamine produced by two different routes and comparing the products. One route was a (Reference Synthesis) involving reaction of benzaldehyde and 2-fluoroethylamine hydrochloride. The other was a “test synthesis” involving reaction of [¹⁸F]Fluoroacetaldehyde and benzylamine.

### Reference Synthesis

A solution of 1 g (10 mmol) of 2-fluoroethylamine hydrochloride in 6 ml. of methanol was prepared in a 25 ml. round bottom flask. Potassium hydroxide (160 mg) was added in one portion to the magnetically stirred solution. When it was completely dissolved, 0.92 ml. (9 mmole) of benzaldehyde was added in one portion. The resulting mixture was stirred at room temperature for 15 minutes before a solution of 200 mg (3.18 mmole) of sodium cyanoborohydride in 2 ml. methanol was added dropwise. After the addition was complete stirring was continued for 30 minutes. Potassium hydroxide (600 mg) was then added, and stirring was continued until it was completely dissolved.

The reaction mixture was filtered through a PTFE membrane syringe filter, Acrodisc®, and the methanol evaporated with a rotary evaporator at 50°. The oil was dissolved in chloroform and purified on silica column. Elution with chloroform-2% methanol-0.5% NEt₃ followed by evaporation of the collected fraction afforded 0.743 g, 54% yield, of N-(2-Fluoroethyl)-benzylamine as an oil.

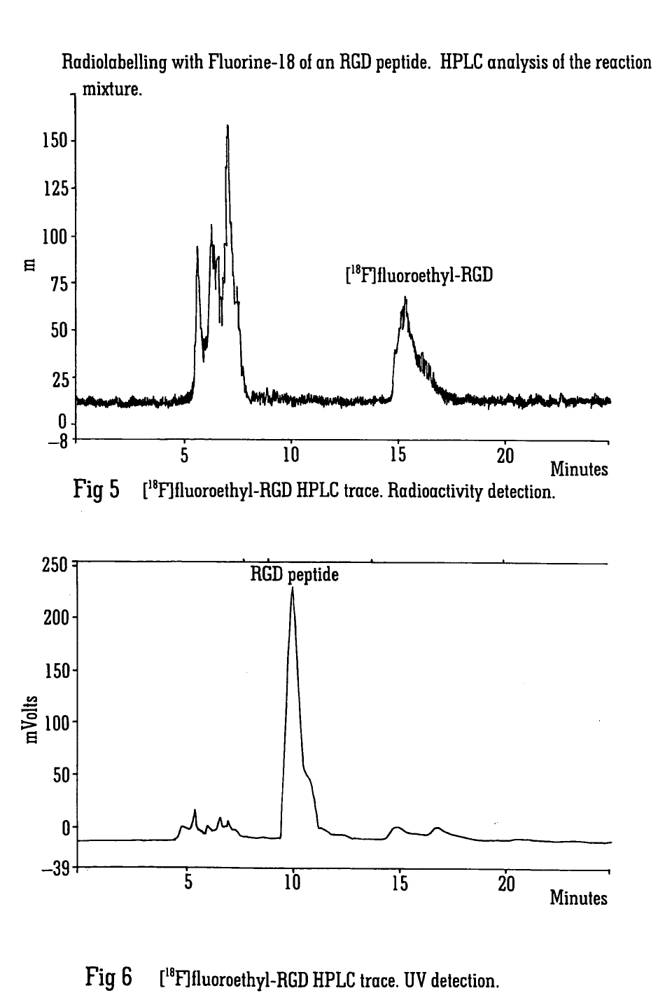

HPLC analysis with water-acetonitrile (70:30 v/v) mixture containing 0.1% triethylamine as eluent and a flow rate of 6 mL/min. gave a retention time of 15 min (FIG. 5). ESI-MS: m/z=154.18 [M+H]⁺. ¹H NMR (CD₃COCD₃) δ: 2.81 (dt, 2H, J=25.7, 4.6 Hz), 3.71 (s, 2H, ArCH₂NH), 4.55 (dt, 2H, J=47.1, 4.7 Hz), 7.25 (t, 1H, J=6.8 Hz), 7.33 (t, 1H, J=7.1 Hz), 7.43 (d, 2H, J=7.3 Hz). ¹⁹F-NMR (CD₃COCD₃) δ: −144.84.

### Test Synthesis

[¹⁸F]N-(2-Fluoroethyl)-benzylamine. [¹⁸F]Fluoroacetaldehyde was trapped at room temperature in 80 μL of cyanoborohydride coupling buffer (sodium cyanoborohydride content 3.8 μmol) to which benzylamine 20 μL of a 0.5 molar solution in acetonitrile were added. The reaction mixture was then heated 20 min at 50° C. before being analysed by HPLC with water-acetonitrile (70:30 v/v) mixture containing 0.1% triethylamine as eluent and a flow rate of 6 mL/min (As shown in FIG. 1).

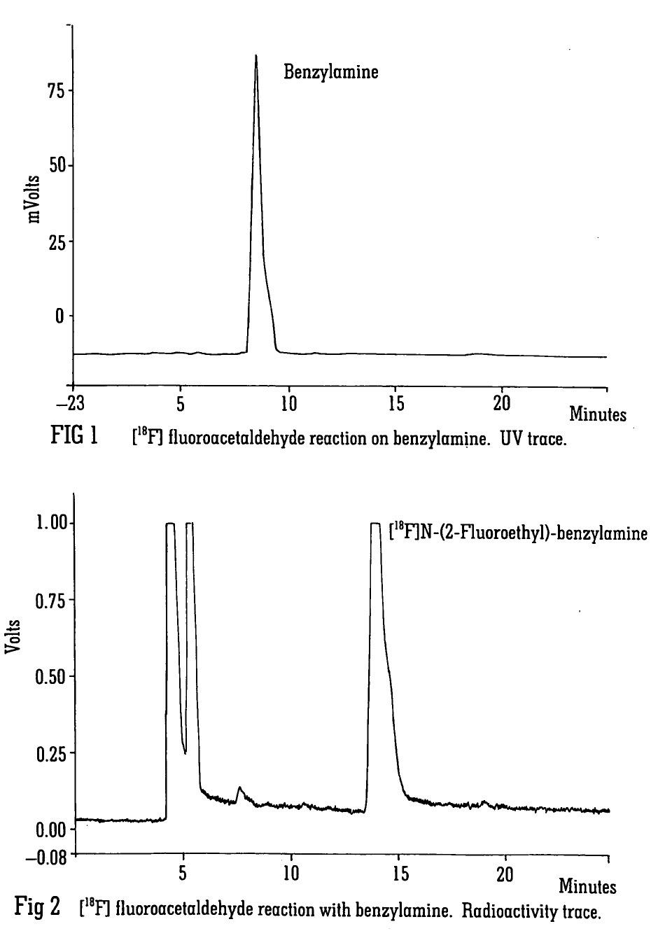

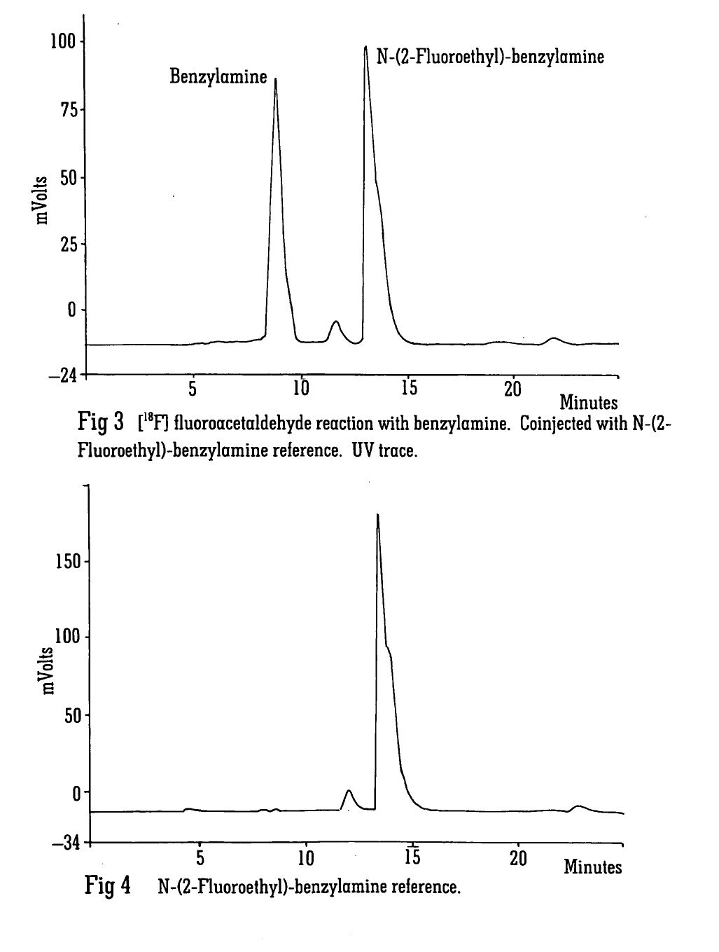

The radioactivity trace (as shown in FIG. 2) showed three radioactive peaks, a first one at 4.2 minutes that represented the unreacted [¹⁸F]fluoroacetaldehyde, a second one at 5.3 minutes that was most likely to be [¹⁸F]fluoroethanol and the third one at 15 minutes co-eluted with stable N-(2-Fluoroethyl)-benzylamine (as shown in FIG. 3) and was identified as [¹⁸F]N-(2-Fluoroethyl)-benzylamine.

The radioactive fractions were collected and the radioactivity measured. [¹⁸F]N-(2-Fluoroethyl)-benzylamine was produced with an overall radiochemical yield of 23% (not corrected for decay).

HPLC analyses of the reaction mixture showed no higher molecular weight products coming from reaction of further oxidation products of excess ethylene di(p-toluenesulfonate), such as glyoxal, with benzylamine (as shown in FIG. 1).

[¹⁸F]N-(2-Fluoroethyl)-RGD peptide. [¹⁸F]Fluoroacetaldehyde was trapped as above in the coupling solution containing 3.1 mg (5 μmol) of cyclic RGDyK peptide. The reaction mixture was then heated 20 min at 50° C. and analysed by HPLC with phosphate buffer (pH 7.4)-acetonitrile (70:30) as eluent. A flow rate of 6 mL/min gave a retention time of 9.8 min. for the c(RGDyK) peptide (as shown in FIG. 6) and 15 min. for the [¹⁸F]N-(2-Fluoroethyl)-c(RGDyK) peptide (as shown in FIG. 5).

### EXAMPLE 2

### Radiolabelling of an Apoptosis Biomarker with [¹⁸F]fluoroacetaldehyde

A gel was produced by addition of the apoptosis biomarker (1 mg) in solution in 50 μl of citrate buffer (citric acid, ˜0.060 M, sodium hydroxide, ˜0.16 M, pH=6) and 10 μl of a 1 M solution of sodium cyanoborohydride in citrate buffer onto the dry polysaccharide (Sephadex® G-75, 4.5 mg).

[¹⁸F]fluoroacetaldehyde was distilled onto the cross-linked polysaccharide gel containing the apoptosis biomarker and sodium cyanoborohydride (reducing agent).

After 8 minutes all of the [¹⁸F]fluoroacetaldehyde was distilled, as monitored by a radioacte detector. The reaction vial was then heated at 37° C. for 45 min after which phosphate buffered saline (1 ml, pH 7.2) was added to the reaction mixture using a programmable syringe pump for infusion and withdrawal. The suspension was then withdrawn and injected into a HPLC, by the syringe pump via a low protein binding filter for purification.

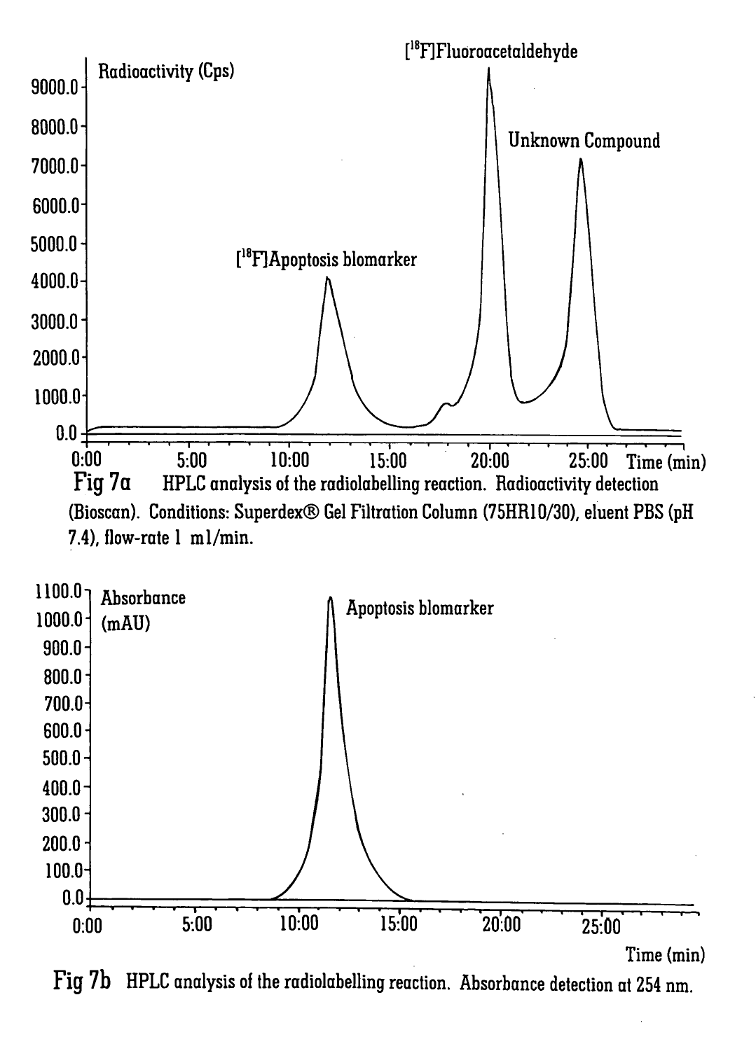

FIG. 7a shows the results of the HPLC analysis of the radiolabelling reaction using radioactivity detection.

For comparison, FIG. 7b shows the HPLC analysis of the radiolabelling reaction using U.V. detection at 254 nm.

### EXAMPLE 3

### Radiolabelling of an Anti-VEGF Antibody with [¹⁸F]fluoroacetaldehyde

A gel was made by addition of 20 μl (0.5 mg) of the solution of antibody formulated for infusion, and 40 μl of a 0.25 M solution of sodium cyanoborohydride in citrate buffer (citric acid, ˜0.060 M, sodium hydroxide, ˜0.16 M, pH=6), onto dry polysaccharide (Sephadex® G-75, 4.5 mg).

[¹⁸F]fluoroacetaldehyde was then distilled onto the polysaccharide (Sephadex® G-75) gel.

After 8 minutes all the [¹⁸F]fluoroacetaldehyde was distilled, as monitored by a radioactivity detector. The reaction vial was then heated at 37° C. for 45 min after which phosphate buffered saline (1 ml, pH 7.2) was added to the reaction mixture using a programmable syringe pump for infusion and withdrawal. The suspension was then withdrawn and injected into a HPLC, by the syringe pump for purification via a low-protein-binding filter for purification.

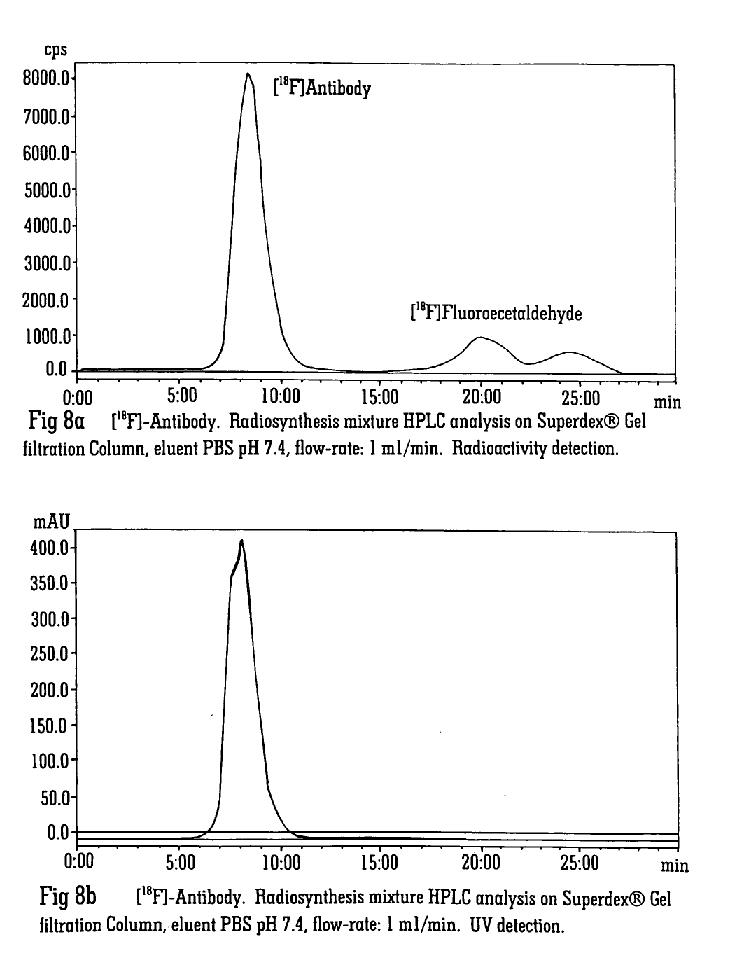

FIG. 8a shows the results of the HPLC analysis of the radiolabelling reaction using radioactivity detection.

For comparison, FIG. 8b shows the HPLC analysis of the radiolabelling reaction using U.V. detection at 254 nm.

### EXAMPLE 4

### ELISA Assay of fluorine-18 Labelled Anti-VEGF Antibody

The radiolabelled anti-VEGF antibody produced as in Example 3 was used as the detection antibody in a VEGF ELISA (ex-R+D), using varying levels of recombinant VEGF.

The results are shown in the following Table:

| Antibody | | Radiolabelled Antibody | | |
|---|---|---|---|---|
| Standard Curve | Average | Standard Curve | Average | |
| (rVEGF): | Absorbance | (rVEGF): | Absorbance | Ratio |
| 2 | 1.133 | 2 | 0.816 | 72% |
| 1 | 0.636 | 1 | 0.444 | 70% |
| 0.5 | 0.311 | 0.5 | 0.244 | 78% |
| 0.25 | 0.177 | 0.25 | 0.129 | 73% |
| 0.125 | 0.080 | 0.125 | 0.071 | 89% |
| 0.0625 | 0.039 | 0.0625 | 0.039 | 100% |
| 0.03125 | 0.022 | 0.03125 | 0.023 | 102% |
| 0 | 0.000 | 0 | 0.000 | |

The results demonstrate that the radiolabelled material was still able to bind to VEGF, and gave ˜70-80% of the signal of the unlabelled antibody. This means that the radiolabelling process does not destroy the ability of the antibody to bind VEGF, although we are unable to say what proportion of the signal (if any) is due to the radiolabelled compound.

### EXAMPLE 5

### Radiolabelling of a 17 KD a peptide with [¹⁸F]fluoroacetaldehyde

A gel was produced made by addition of 20 μl (2 mg) of a solution of the 17 KDa peptide formulated for injection, and 40 μl of a 0.25 M solution of sodium cyanoborohydride in citrate buffer (citric acid, ˜0.060 M, sodium hydroxide, ˜0.16 M, pH=6), onto dry polysaccharide (Sephadex® G-75, 4.5 mg).

[¹⁸F]fluoroacetaldehyde was distilled into a polysaccharide (Sephadex® G-75) gel.

After 8 minutes all the [¹⁸F]fluoroacetaldehyde was distilled, as monitored by a radioactivity detector. The reaction vial was then heated at 37° C. for 45 min after which phosphate buffered saline (1 ml, pH 7.2) was added to the reaction mixture using a programmable syringe pump for infusion and withdrawal. The suspension was then withdrawn and injected into a HPLC, by the syringe pump for purification via a low-protein-binding filter for purification.

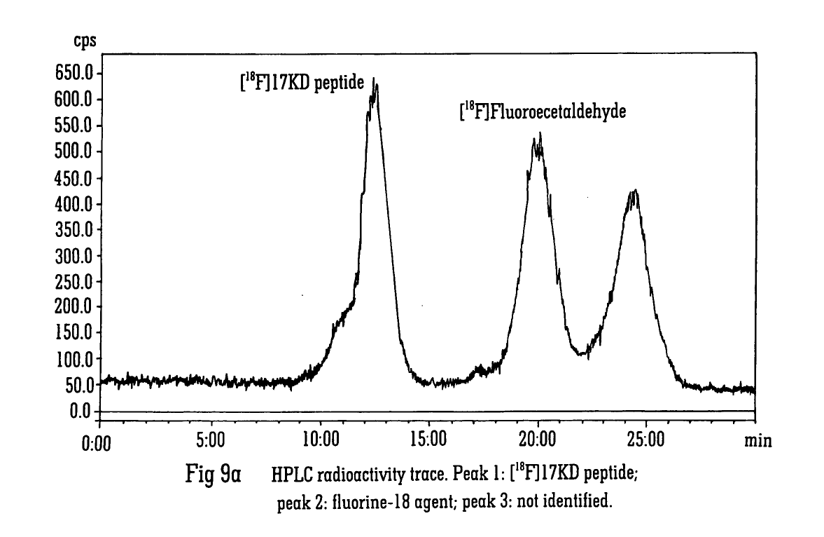

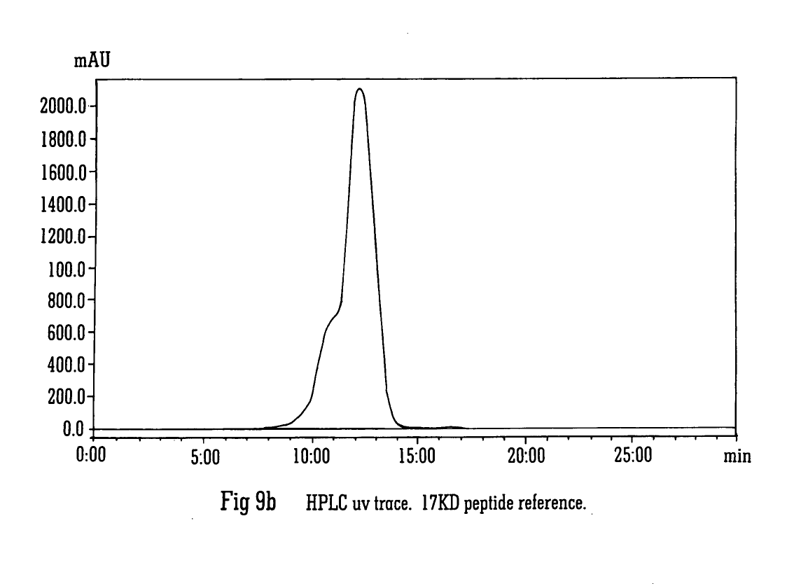

FIG. 9a shows the results of the HPLC analysis of the radiolabelling reaction using radioactivity detection.

For comparison, FIG. 9b shows the HPLC trace obtained for the non-labelled 17 Kda peptide formulated for injection using absorbance detection at 254 nm.

### EXAMPLE 6

### Binding Assay of the 17 Kd a Peptide

An in vitro assay was performed to investigate the ability of the radiolabelled 17 kDa protein produced in Example 5 to bind to its cellular receptor. The assay was conducted using rat brain slices, which contain receptors for the 17 kDa protein. Binding of the labelled protein was detected by autoradiography.

Specific binding of the protein to its receptor was determined by comparing the level of total binding (which includes both specific and non-specific binding) and the level of non-specific binding in matched brain sections. This allowed discrimination between saturable specific binding of the labelled 17 kDa protein to its cellular receptor, and non-saturable non-specific binding of the protein to constituents other than its cellular receptor. Specific binding was derived by subtraction of the non-specific binding from the total binding value.

To determine total binding of the labelled 17 kDa protein, slices of healthy rat brains, were bathed in solutions of varying concentrations 5 to 50 nm of the radiolabelled 17 kDa protein. After 30 minutes incubation the slices were washed in water, to remove unbound radiolabelled protein, before performing autoradiography.

To determine non-specific binding brain slices treated as above were further washed with solutions containing a large excess of non-radiolabelled 17 kDa protein at varying concentrations (5 to 50 μm) before performing autoradiography. The non-labelled protein was able to compete with the radiolabelled protein for specific binding to cellular receptors present in the brain sections. Radiolabelled protein that could not be displaced by increasing concentrations of non-radiolabelled protein could thus be deduced to be non-specifically bound.

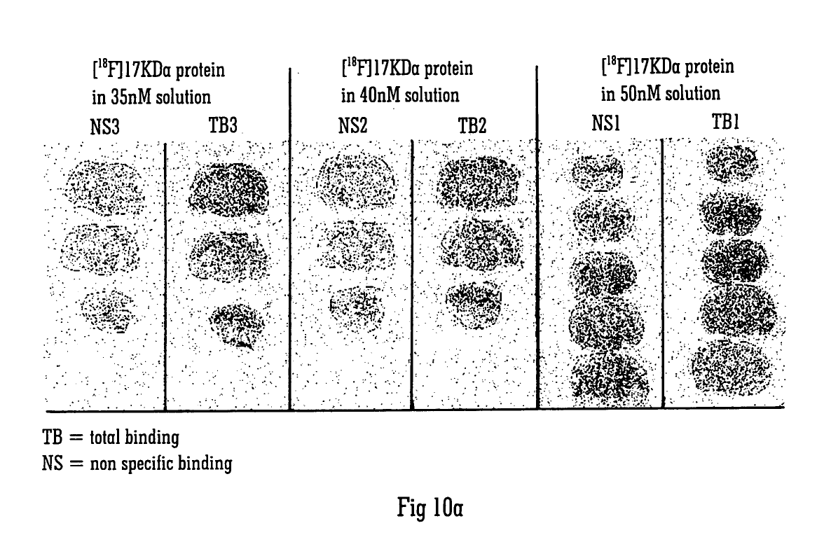

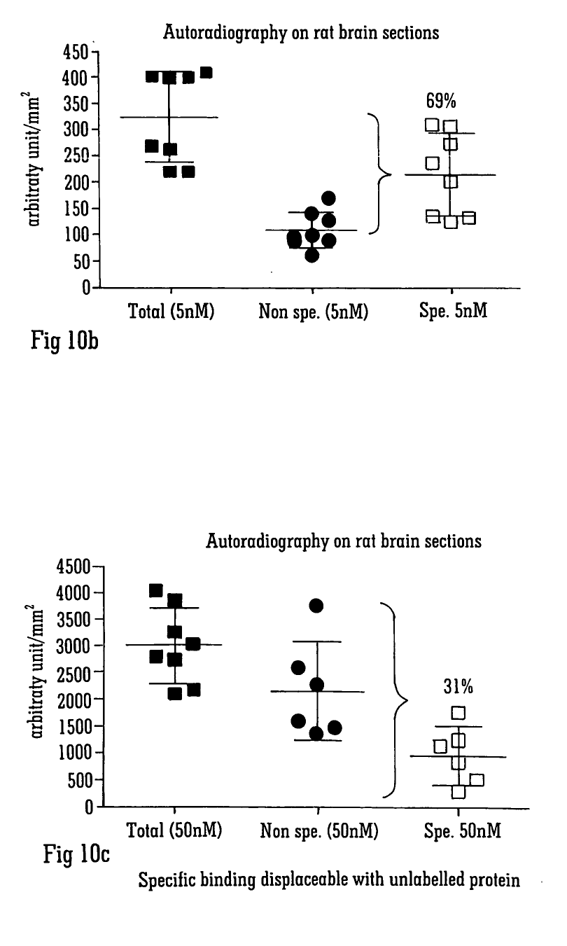

Examples of autoradiographies illustrating total binding and non-specific (NS) binding of the radiolabelled 17 kDa protein are shown in FIG. 10. Comparison of the total binding and non-specific indicates that specific binding of the radiolabelled 17 kDa protein to its cellular receptor accounts for roughly 40-50% of total binding, illustrating that receptor binding is not adversely effected by the radiolabelling process.

All documents and other information sources cited above and below are hereby incorporated in their entirety by reference.

### REFERENCES

Albright J. D., Goldman L., 1967. Dimethyl sulfoxide-acid anhydride mixtures for the oxidation of alcohols. J. Am. Chem. Soc. 89, 2416-2423.
Anderson G. J., 1959. A new and selective method of oxidation. The conversion of alkyl halides and alkyl tosylates to aldehydes. J. Am. Chem. Soc. 81, 4113-4114.
Berger G., Maziere M., Prenant C., Sastre J., Comar D., 1984. 11C Labelling of a protein: Concanavalin A. Int. J. Appl. Radiat. Isot. 35, 81-83.
Block D., Coenen H. H., Stockling G., 1986. NCA 18F-fluoroalkylation of H-acidic compounds. J. Label. Compd. Radiopharm. 25, 201-215.
Borch R. F., Bernstein M. D., Durst H. D., 1971. The cyanohydridoborate anion as a selective reducing agent. J. Am. Chem. Soc. 93, 2897-2904.
Borch R. F., Dusta H. D., 1969. Lithium Cyanohydridoborate, a Versatile New Reagent. J. Am. Chem. Soc. 91, 3996-3997.
Cai W., Zhang X., Wu Y. and Chen X., 2006. A Thiol-Reactive ¹⁸F-Labeling Agent, N-[2-(4-18F-Fluorobenzamido)Ethyl]Maleimide, and Synthesis of RGD Peptide-Based Tracer for PET Imaging of αvβ3 Integrin Expression. J Nucl Med. 47, 1172-1180.

Chen X., Park R., Shahinian A. H., Tohme M., Khankaldyyan V., Bozorgzadeh M. H., Bading J. R., Moats R., Laug W. E., Conti P. S., 2004. 18F-labeled RGD peptide: initial evaluation for imaging brain tumor angiogenesis. Nucl. Med. Bio. 31, 179-189.
Gautier E. C. L., Graham A. E., McKillop A., Standen S. P., Taylor R. J. K., 1997. Acetal and ketal deprotection using montmorillonite K10: The first synthesis of syn-4,8-dioxatricyclo[5.1.0.03,5]-2,6-octanedione. Tetrahedron Lett. 38, 1881-1884.
Haubner R., Wester H. J., Weber W. A., Mang C., Ziegler S. I., Goodman S. L., Senekowitsch-Schmidtke R., Kessler H., Schwaiger M., 2001. Noninvasive imaging of αvβ3 integrin expression using ¹⁸F-labeled RGD-containing glycopeptide and positron emission tomography. Cancer. Res. 61, 1781-5.
Haubner R., Kuhnast B., Mang C., Weber W. A., Kessler H., Wester H. J., Schwaiger M., 2004. [18F]Galacto-RGD: Synthesis, Radiolabelling, Metabolic Stability, and Radiation Dose Estimates. Bioconjugate Chem. 15, 61-69.
Komblum W., Jones W. J., Anderson G. J., 1959. A new and selective method of oxidation. The conversion of alkyl halides and alkyl tosylates to aldehydes. J. Am. Chem. Soc. 81, 4113-4114.
Means G. E., Feeney R. E., 1968. Reductive alkylation of amino groups in proteins. Biochemistry 7, 2192-2201.
Means G. E., 1977. Reductive alkylation of amino groups. Methods Enzymol. 47, 469-478.
Ogawa M., Hantaro K., Kawasumi Y., Fuji N., Kawaguchi M., Doi R., Imamura M., Yamamoto M., Ajito K., Mukai T., Saji H., Ito K., 2003. Direct electrofluorination of a cyclic RGD peptide for in vivo αᵥβ₃ integrin related tumor imaging. Nucl. Med. Bio. 30, 1-9.
Omura K., Swern D., 1978. Oxidation of alcohols by “activated” dimethyl sulfoxide. a preparative, steric and mechanistic study. Tetrahedron 34, 1651-1660.
Pfitzner K. E., Moffatt J. G., 1965. Sulfoxide-Carbodiimide Reactions. J. Am. Chem. Soc. 87, 5661-5669.
Poethko T., Schottelius M., Thumshirn G., et al., 2004. Two-Step Methodology for High-Yield Routine Radiohalogenation of Peptides: 18F-Labeled RGD and Octreotide Analogs. J. Nucl. Med. 45, 892-902.
Rice R. H., Means G. E., 1971. Radioactive Labelling of Proteins in Vitro. J. Biol. Chem. 246, 831-832.
Straatmann M. G., Welch M. J., 1975. A general method for labelling proteins with 11C. J. Nucl. Med. 16, 425-428.
Villemin D. and Hammadi M., 1995. Oxidation by DMSO II. An efficient Synthesis of α,β-diketones from α,β-dibromides. Synth. Commun. 25, 3145-3148.
Zijlstra S, Gunawan J, Burchert W, 2003. Synthesis and evaluation of a ¹⁸F-labelled recombinant annexin-V derivative, for identification and quantification of apoptotic cells with PET. Appl. Radiat. Isot. 58, 201-207.

1. Compounds of the formula (I):
¹⁸F—(CHR)ₙ(CH₂)ₘCHO (I)
in which n and m are independently 0 and 1 with at least one of n and m being 1, and R (if present) is a hydrogen atom or a methyl group, subject to the proviso that if n is 1 and R is methyl then m is 0.

2. A compound as claimed in claim 1 wherein n is 0.

3. A method of synthesising an aldehyde of the formula (I):
¹⁸F—(CHR)ₙ(CH₂)ₘCHO (I)
in which n and m are independently 0 and 1 with at least one of n and m being l, and R (if present) is a hydrogen atom or a methyl group, subject to the proviso that if n is 1 and R is methyl then m is 0, the method comprising the steps of:
(a) providing a compound of the formula (II).
¹⁸F—(CHR)ₙ(CH₂)ₘ-L (II)
in which m, n and R are as defined above and -L represents a leaving group;
(b) oxidising the compound of formula (II) to the aldehyde (I); and
(c) recovering the aldehyde (I) from the reaction mixture.

4. A method as claimed in claim 2 wherein n is 0.

5. A method as claimed in claim 2 wherein the wherein the compound of formula (II) is prepared by reaction of the ¹⁸F— anion with a compound of the formula (III):
L—(CHR)ₙ(CH₂)ₘ-L (III)
in which m, n, L and R are as defined above.

6. A method as claimed in claim 5 in which -L is a tosylate group.

7. A method as claimed in claim 5 wherein the ¹⁸F⁻ anion is provided by a salt of the formula M¹⁸F, where M is an alkali metal, complexed with a chelating agent and the reaction of the alkali metal salt with the compound of formula (II) is effected in a polar organic medium in which the alkali metal salt is dissolved.

8. A method as claimed in claimed in claim 7 wherein the chelating agent is a cryptand.

9. A method as claimed in claim 6 wherein the cryptand is an aminopolyether.

10. A method as claimed in claim 6 wherein the polar organic medium is acetonitrile.

11. A method as claimed in claim 6 wherein oxidation of compound (II) is effected by heating the compound in Dimethylsulfoxide (DMSO).

12. A method as claimed in claim 6 wherein step (c) is effected by distilling the compound of formula (I) from the product mixture obtained in step (b).

13. A method of synthesising an aldehyde of the formula (I):
¹⁸F—(CHR)ₙ(CH₂)ₘCHO (I)
in which n and m are independently 0 and 1 with at least one of n and m being 1, and R (if present) is a hydrogen atom or a methyl group, subject to the proviso that if n is 1 and R is methyl then m is 0, the method comprising the steps of:
(i) reacting a complex of M¹⁸F, where M is an alkali metal, and a crytand dissolved in acetonitrile with a compound of the formula (IIIa)
Ts-O—(CHR)ₙ(CH₂)ₘ—O-Ts (IIIa)
in which Ts-O— represents a tosylate group so as to produce an intermediate of formula (IIa):
¹⁸F—CH₂—(CH₂)ₙCH₂—O-Ts (IIa);
(ii) evaporating the acetonitrile from the intermediate of formula (IIa);
(iii) oxidising the intermediate of formula (IIa) from which the acetonitrile has been removed by heating in anhydrous dimethylsulfoxide to produce the aldehyde (I); and
(iv) distilling the aldehyde (I) from the reaction mixture.

14. A method as claimed in claim 3 wherein L is of the formula:

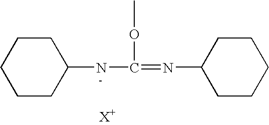

15. A method of preparing a radiolabelled adduct of a compound containing a free primary or secondary amino group, the method comprising reacting said compound with an aldehyde of the formula:
¹⁸F—(CHR)ₙ(CH₂)ₘCHO (I)
in which n and m are independently 0 and 1 with at least one of n and m being 1, and R (if present) is a hydrogen atom or a methyl group, subject to the proviso that if n is 1 and R is methyl then m is 0 under conditions in which the amino group of the compound undergoes a condensation reaction with the —CHO group of the aldehyde of formula (I) to produce the adduct.

16. A method as claimed in claim 15 wherein n is 0.

17. A method as claimed in claim 15 wherein said compound containing a free primary or secondary amino group is contained within a gel.

18. A method as claimed in claim 17 wherein the gel is an aqueous gel.

19. A method as claimed in claim 17 wherein the gel is comprised of cross-linked polysaccharides.

20. A method as claimed in claim 17 wherein the aldehyde is distilled onto the gel.

21. A method as claimed in claim 15 effected under reductive amination conditions.

22. A method as claimed in claim 21 wherein the compound containing the free amino group is selected from the group consisting of peptides, proteins, antibodies, antibody fragments and oligonucleotides.

23. The use of a radiolabelled adduct prepared by the method of claim 15 for PET imaging.
